(19) United States
(12) Patent Application Publication
(10) Pub. No.: US 2008/0268036 A1
(43) Pub. Date: Oct. 30, 2008

(54) Co-processing of active pharmaceutical/nutraceutical ingredients

(75) Inventors: Alen Guy, Bethel, CT (US); David Schaible, Ulster Park, NY (US); Theodore Montalto, New Haven, CT (US)

Correspondence Address: Davidson, Davidson & Kappel, LLC, 485 7th Avenue, 14th Floor, New York, NY, 10018, US

(73) Assignee: JRS Pharma, Patterson, NY (US)

(21) Appl. No.: 12/150,282

(22) Filed: Apr. 24, 2008

Related U.S. Application Data
(60) Provisional application No. 60/926,158, filed on Apr. 24, 2007.

(51) Int. Cl. A61K 9/48; A61K 9/14; A61K 31/5415; A61K 31/715; A61K 31/7008; A61K 9/20

(52) U.S. Cl. 424/451; 424/489; 514/226.5; 514/54; 514/62; 424/464; 424/468

(57) ABSTRACT

A process for preparing agglomerated particles comprising a) preparing a slurry of a pre-manufactured agglomerated particles consisting of microcrystalline cellulose and one or more compressibility augmenting agents, and an active ingredient; and b) drying the slurry to form active agent agglomerated particles.

This application claims the benefit of U.S. Provisional Patent Application No. 60/926,158, filed on Apr. 24, 2007, the disclosure of which is hereby incorporated by reference in its entirety.

### BACKGROUND

Spray dryers are well known in the art for drying pharmaceutical and nutraceutical active agents and excipients. In general, a spray dryer is used to process fluid materials into powders. Typically, the fluid material is introduced into the spray dryer in the form of a solution, suspension, emulsion, slurry, dispersion or thin paste. In operation, the fluid material is fed from a feed delivery system to an atomizer. The atomizer disperses the fluid material into the drying chamber in fine droplets. A heated air supply applies heated air to the fine droplets in the drying chamber, causing the fine droplets to be dried into a powder, the powder being collected in a collection system.

Spray dryers are widely used in the preparation of active agents. For example, it is known to spray dry an active agent in the form of a fluid material (for example, a liquid herbal extract) to form a powder, and thereafter, to blend the powder with conventional tableting agents, and then compress the resulting mixture into a tablet.

Examples of such tableting agents include lubricants, diluents, binders, disintegrants, and direct compression vehicles. Lubricants are typically added to avoid the material(s) being tableted from sticking to the punches. Commonly used lubricants include magnesium stearate, stearic acid, sodium stearyl fumarate, hydrogenated vegetable oil, and calcium stearate. Such lubricants are commonly included in the final tableted product in amounts of less than 1% by weight. Diluents are frequently added in order to increase the bulk weight of the material to be tableted in order to make the tablet a practical size for compression. This is often necessary where the dose of the drug is relatively small. Binders are agents which impart cohesive qualities to the powdered material(s). Commonly used binders include starch, and sugars such as sucrose, glucose, dextrose, and lactose. Typical disintegrants include starch derivatives and salts of carboxymethylcellulose. Direct compression vehicles include, for example, processed forms of cellulose, sugars, and dicalcium phosphate dihydrate, among others. Microcrystalline cellulose is an example of a processed cellulose that has been utilized extensively in the pharmaceutical industry as a direct compression vehicle for solid dosage forms.

Silicified microcrystalline cellulose is a particularly useful direct compression vehicle. Silicified microcrystalline cellulose is a particulate agglomerate of coprocessed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (μm), based on average primary particle size. Preferably, the silicon dioxide comprises from about 0.5% to about 10% of the silicified microcrystalline cellulose, and most preferably from about 1.25% to about 5% by weight relative to the microcrystalline cellulose. Moreover, the silicon dioxide preferably has a particle size from about 5 nm to about 40 μm, and most preferably from about 5 nm to about 50 μm. Moreover, the silicon dioxide preferably has a surface area from about 10 m²/g to about 500 m²/g, preferably from about 50 m²/g to about 500 m²/g, and more preferably from about 175 m²/g to about 350 m²/g. Silicified microcrystalline cellulose, and methods for its manufacture, are described in U.S. Pat. No. 5,585,115, the entire disclosure of which is hereby incorporated by reference. Silicified microcrystalline cellulose is commercially available from JRS Pharma, LP (formerly available from Penwest Pharmaceuticals, Inc.), under the trademark Prosolv®. Prosolv® is available in a number of grades, including, for example, Prosolv® SMCC 50, Prosolv® SMCC 90, Prosolv® SMCC HD90, and Prosolv® SMCC 90LM.

### SUMMARY OF THE INVENTION

It is an object of the present invention to provide formulation of co-processed active agent agglomerated particles with high functionality characteristics (e.g., flow improvements, hygroscopicity, compactibility, content uniformity).

It is another object of the present invention to provide a formulation of co-processed active agent particles that is directly compressible.

It is another object of the present invention to provide a formulation of co-processed active agent particles such that the active agent particles are highly effective carrier/binder.

It is another object of the present invention to provide a formulation of co-processed active agent particles wherein the flow and content uniformity of the active agent particles is maximized.

It is an object of the present invention to provide a method of manufacturing active agent agglomerated particles.

It is another object of the present invention to provide a method of manufacturing a solid dosage form comprising active agent agglomerated particles.

Another object of the present invention is directed to a Piroxicam solid dosage prepared by the methods described herein.

Another object of the present invention is directed to a Glucosamine dosage form prepared by the methods described herein.

Another object of the present invention is directed to a Ramipril dosage form prepared by the methods described herein.

It is another object of the present invention to provide methods of preparing active agent agglomerated particles for use in preparing solid dosage forms suitable for treatment of orthopedic disorders and conditions and cardiac disease.

In accordance with one embodiment, the present invention is directed to a method of preparing a pharmaceutical formulation, comprising a) preparing a slurry of pre-manufactured agglomerated particles consisting of microcrystalline cellulose and one or more compressibility augmenting agents, and an active agent; and b) drying the slurry to form active agent agglomerated particles.

In accordance with another embodiment, the present invention is directed to method of preparing a pharmaceutical formulation, comprising a) preparing an aqueous slurry of microcrystalline cellulose, a compressibility augmenting agent and other, optional, pharmaceutically acceptable excipients; b) drying the mixture of ingredients prepared in step a) in a manner which inhibits quasi-hornification of the microcrystalline cellulose to obtain agglomerate particles; c) preparing a slurry containing the agglomerated particles obtained in step b) together with a suitable amount of an active agent, and other, optional, pharmaceutically acceptable excipients; and; d) drying the slurry to form active agent agglomerated particles.

In another embodiment, the present invention is directed to a method of preparing a pharmaceutical formulation, comprising a) preparing a slurry of pre-manufactured agglomerated particles consisting of microcrystalline cellulose and one or more compressibility augmenting agents; and b) combining dry active agent particles and the slurry in a dryer to form active agent agglomerated particles.

In another embodiment, the present invention is directed to a method of preparing a pharmaceutical formulation, comprising a) preparing a slurry of pre-manufactured agglomerated particles consisting of silicified microcrystalline cellulose and an active agent; and b) drying the slurry to form active agent agglomerated particles.

In yet another embodiment, the present invention is directed to a method of preparing a pharmaceutical formulation, comprising a) preparing a slurry of microcrystalline cellulose and an active agent; and b) drying the slurry to form active agent agglomerated particles.

In yet another embodiment, the present invention is directed to solid dosage forms formed by a) preparing a slurry of pre-manufactured agglomerated particles consisting of microcrystalline cellulose and one or more compressibility augmenting agents, and an active agent; b) drying the slurry to form active agent agglomerated particles; and c) incorporating the active agent agglomerated particles into a dosage form.

In another embodiment, the present invention is directed to solid dosage forms formed by preparing a pharmaceutical formulation, comprising a) preparing an aqueous slurry of microcrystalline cellulose, a compressibility augmenting agent and other, optional, pharmaceutically acceptable excipients; b) drying the mixture of ingredients prepared in step a) in a manner which inhibits quasi-hornification of the microcrystalline cellulose to obtain agglomerate particles; c) preparing a slurry containing the agglomerated particles obtained in step b) together with a suitable amount of an active agent, and other, optional, pharmaceutically acceptable excipients; d) drying the slurry to form active agent agglomerated particles; and e) incorporating the active agent agglomerated particles into a dosage form.

In another embodiment, the present invention is directed to solid dosage forms formed by a) preparing a slurry of pre-manufactured agglomerated particles consisting of microcrystalline cellulose and one or more compressibility augmenting agents; b) combining dry active agent particles and the slurry in a dryer to form active agent agglomerated particles; and c) incorporating the active agent agglomerated particles into a dosage form.

In another embodiment, the present invention is directed to solid dosage forms formed by preparing a) preparing a slurry of pre-manufactured agglomerated particles consisting of silicified microcrystalline cellulose and an active agent; b) drying the slurry to form active agent agglomerated particles; and c) incorporating the active agent agglomerated particles into a dosage form.

The compressibility augmenting agents utilized in the present invention may (i) physically restrict the proximity of the interface between adjacent cellulose surfaces; or (ii) inhibit interactions between adjacent cellulose surfaces; or (iii) both (1) and (ii)

Compressibility augmenting agents which inhibit surface-to-surface interactions between surfaces of the microcrystalline cellulose include any material which has the ability, via a portion of the molecule, to bind or interact with the surface of the microcrystalline cellulose and at the same time, via another portion of the molecule, to inhibit the attraction of the cellulose surfaces, e.g., via a hydrophobic portion or “tail”. Suitable compressibility augmenting agents will preferably have an HLB value of at least 10, preferably at least about 15, and more preferably from about 15 to about 40 or greater. To date, compressibility augmenting agents which have shown the greatest effect have had relatively high HLB values, and therefore an HLB value from about 30 to about 40 or greater is most preferred. Agents which exhibit these properties include certain surfactants such as sodium lauryl sulfate and polysorbate 40, and highly polar compounds, including pharmaceutically acceptable dyes such as congo red.

In certain embodiments, the active agent itself can be used as the compressibility augmenting agent. When the active agent is utilized as the compressibility augmenting agent, it is preferable to use active agent particles having a small particles size, e.g., submicron or nanoparticles. In this embodiment, the active agent acts similar to, e.g., a colloidal silicon dioxide.

As described in further detail below, the active agent agglomerated particles in accordance with certain embodiments of the present invention described above provide a number of advantages including superior flow characteristics and superior compaction characteristics to prior art compositions. For example, the methods of the present invention may provide co-processed active agent agglomerated particles that allow for the active agent to be directly compressible and a highly effective carrier/binder. The methods may also provide active agent agglomerated particles with enhanced stability and bioavailability of the active agent. As one of ordinary skill in the art will appreciate, the superior compaction characteristics provided by these embodiments of the present invention allow faster and more efficient processing for tablets, and, moreover, allow a larger percentage of active agent to be included in each tablet. The superior compaction characteristics also allow for the manufacture of smaller dosage forms, e.g., tablets, as less excipients may be required, e.g., disintegrants and lubricants. Another advantage of the present invention is the reduction in blending steps, the increase in production capacity without increased costs and improvement of production yields. The inventors of the present invention also found that the formulations prepared utilizing the methods of the present invention may achieve a substantially greater level of content uniformity compared to prior art formulations utilizing agglomerated particles that include microcrystalline cellulose having enhanced compactibility (e.g., ProSolv®). For example, “naked” (non co-processed) piroxicam 10 mg formulations tested had a content uniformity of about 4.3%, whereas co-processed piroxicam 10 mg formulations prepared by the methods of the present invention had a content uniformity of less than about 0.6%. As one of ordinary skill in the art will appreciate, this is advantageous over conventional wet granulation and equivalent to solid dispersion techniques such as hot melt. Previous art has referred to co-spray drying of certain materials as a solid dispersion (Beyerinck, et al.; U.S. Pat. No. 6,763,607).

In certain variants of the embodiments described herein, the active agent is piroxicam and its pharmaceutically acceptable salts, derivatives and mixtures thereof.

In certain variants of the embodiments described herein, the active agent is ramipril and its pharmaceutically acceptable salts, derivatives and mixtures thereof.

In certain other embodiments, the active agent is glucosamine and its pharmaceutically acceptable salts and esters, including, for example, glucosamine, glucosamine HCL, glucosamine SO₄Na, and glucosamine SO₄K. In other variants, the active agent is chondroitin and its pharmaceutically acceptable salts and esters, including chondroitin sulfate. In still other embodiments, the active agent includes both glucosamine and its pharmaceutically acceptable salts and esters and chondroitin and its pharmaceutically acceptable salts and esters.

The term “environmental fluid” is meant for purposes of the invention to encompass, e.g., an aqueous solution, or gastrointestinal fluid.

By “sustained release” it is meant for purposes of the invention that a therapeutically active medicament is released from the formulation at a controlled rate such that therapeutically beneficial blood levels (but below toxic levels) of the medicament are maintained over an extended period of time, e.g., providing a 12 hour or a 24 hour dosage form.

By “primary particle size” it is meant for purposes of the invention that the particles are not agglomerated. Agglomeration is common with respect to silicon dioxide particles, resulting in a comparatively average large agglomerated particle size.

For purposes of the invention that the term “slurry” also includes solutions, suspensions, dispersions, emulsions and the like.

### BRIEF DESCRIPTION OF THE DRAWINGS

FIG. 1 is a block diagram of a spray dryer including a fluid active agent and a source of silicified microcrystalline cellulose.

FIG. 2 is a graph that compares the dissolution profile of a slurry co-processed Piroxicam tablet (SCP 10 mg; 5 kp hardness) prepared with the active agent agglomerated particles of Example 6 and the dissolution profile of a co-processed Piroxicam tablet with sodium lauryl sulfate (CP 10 mg w/SLS; 4 kp hardness) prepared with the active agent agglomerated particles of Example 7 with the dissolution profile of two non-coprocessed piroxicam formulations.

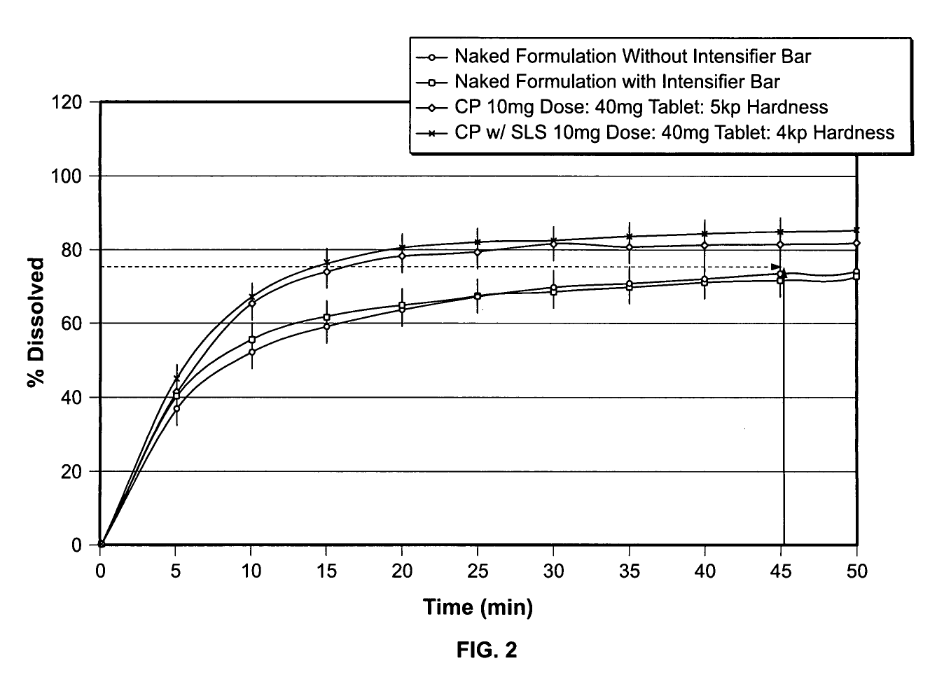

FIG. 3 is a graph that depicts the dissolution profile of two co-processed Piroxicam formulations, wherein the formulation of Trial 3 contains no additional HD ProSolv® added and the formulation of Trial 4 contains additional HD ProSolv® added.

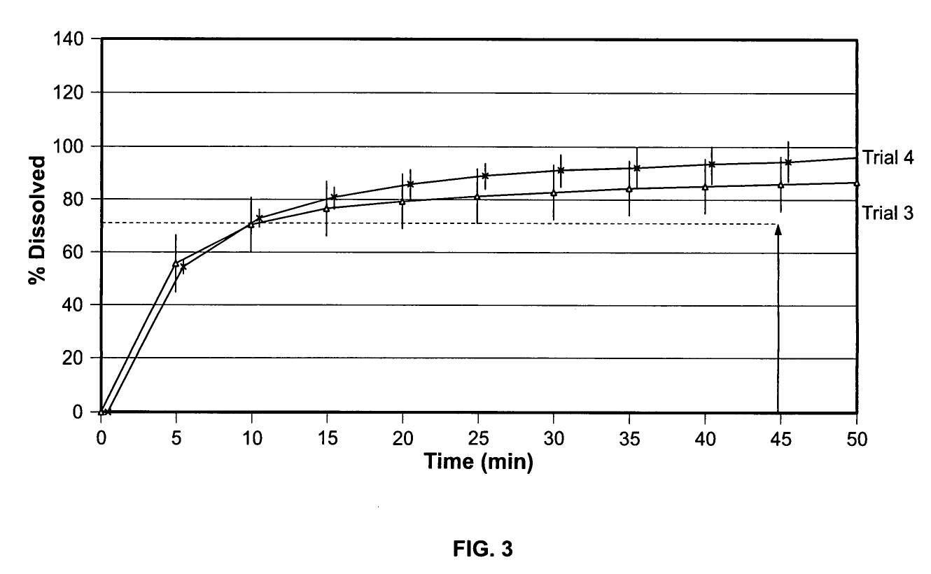

FIG. 4 is a graph that compares the dissolution profile of a slurry co-processed Piroxicam tablet (SCP 10 mg; 5 kp hardness) prepared with the active agent agglomerated particles of Example 6; the dissolution profile of a co-processed Piroxicam tablet with sodium lauryl sulfate (CP 10 mg w/SLS; 4 kp hardness) prepared with the active agent agglomerated particles of Example 7; the dissolution profile of a co-processed Piroxicam tablet without surfactant (CP 10 mg; 10 kp hardness) prepared with the active agent agglomerated particles of Example 4 with the dissolution profile of two non-coprocessed piroxicam formulations.

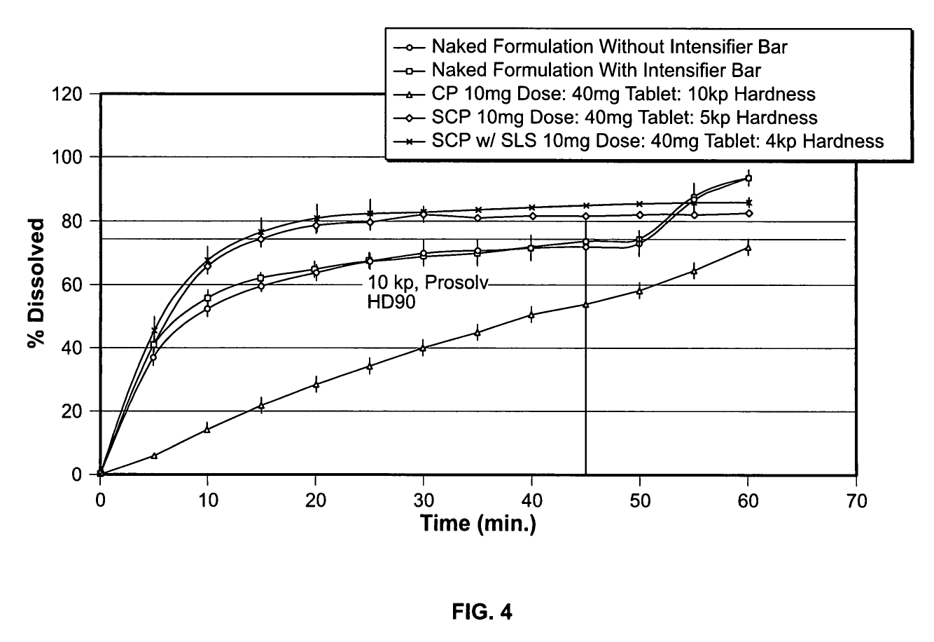

FIG. 5 is a graph that compares the dissolution profile of the non-coprocessed piroxicam formulations, one with sodium lauryl sulfate and the other without sodium lauryl sulfate of Examples 12 and 13.

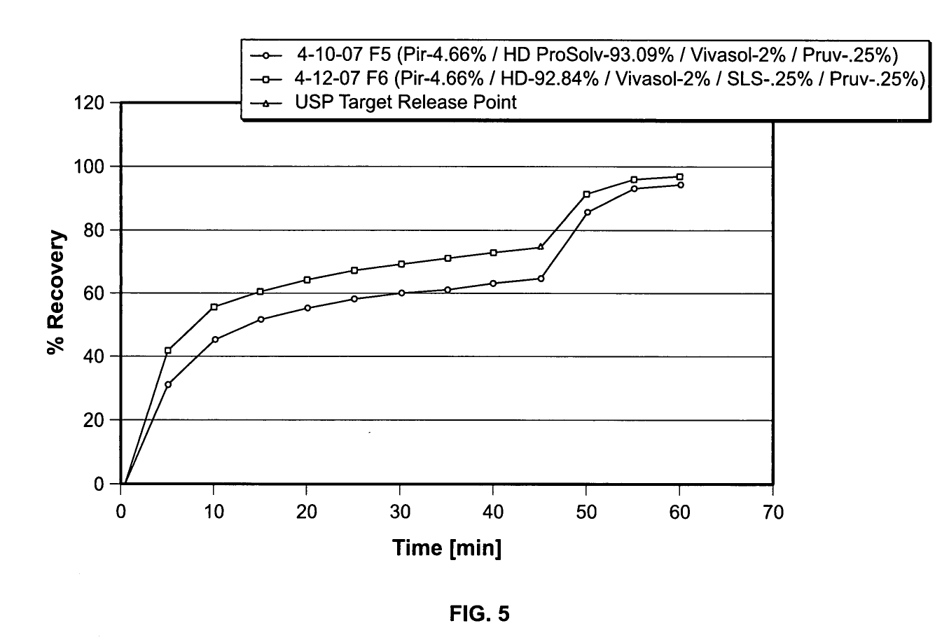

FIG. 6 is a graph that compares the dissolution profile of the coprocessed piroxicam formulations, one with sodium lauryl sulfate and the other without sodium lauryl sulfate of Examples 8 and 9.

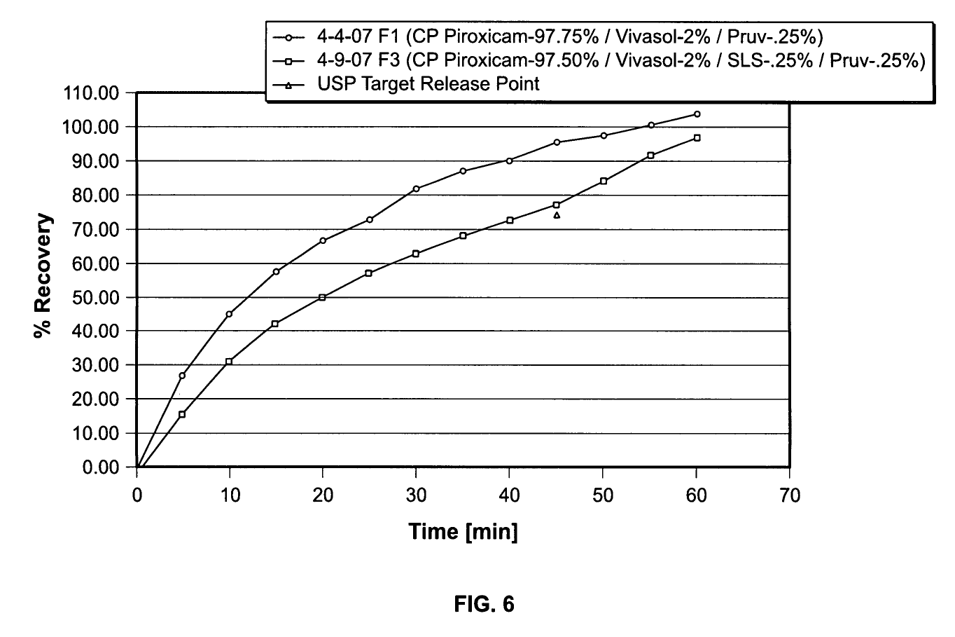

FIG. 7 is a graph that compares the dissolution profile of the coprocessed piroxicam formulations with additional ProSolv®, one with sodium lauryl sulfate and the other without sodium lauryl sulfate of Examples 10 and 11.

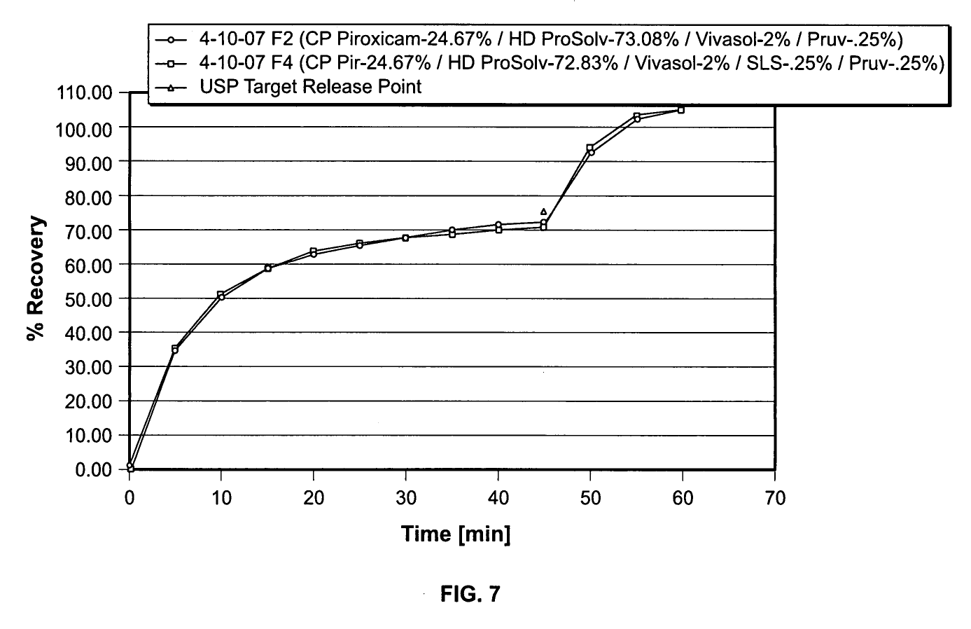

### DETAILED DESCRIPTION

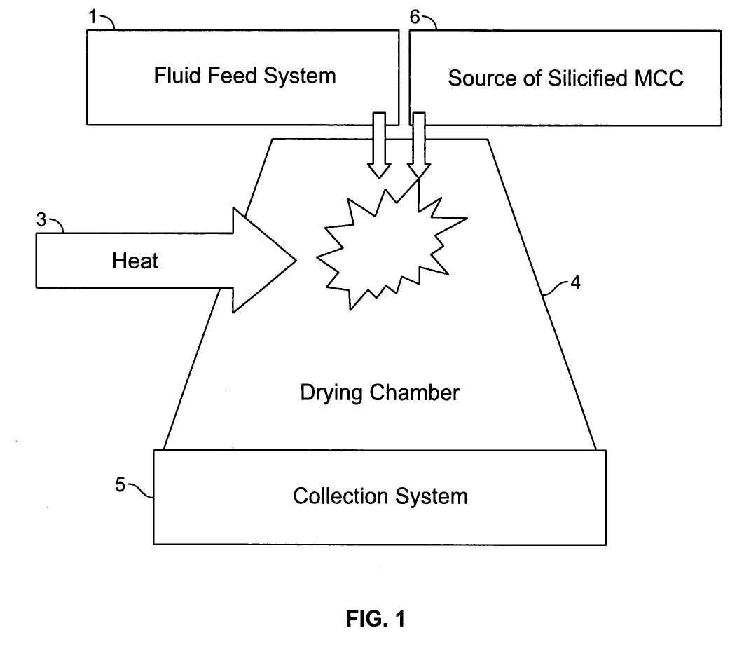

Spray dryers are well known in the art for drying pharmaceutical and nutraceutical active agents and excipients. In general, a spray dryer is used to process fluid materials, e.g., slurries, into powders. Typically, the fluid material is introduced into the spray dryer in the form of a solution, slurry, suspension, emulsion, or thin paste. Referring to FIG. 1, a typical spray dryer including a fluid feed system 1, an atomizer 2, a heated air supply 3, a drying chamber 4, and a collection system 5. In operation, the fluid material is fed from the fluid feed system to the atomizer. The atomizer disperses the fluid material into the drying chamber in fine droplets. The heated air supply applies heated air to the fine droplets in the drying chamber, causing the fine droplets to be dried into a powder, the powder being collected in the collection system. In certain spray dryers, extremely fine particles that float up from the collection system (referred to in the art as “fines”) are recycled back into the path of the atomized fluid material.

In accordance with an embodiment of the present invention, the slurry is a mixture of pre-manufactured agglomerated particles consisting of microcrystalline cellulose and one or more compressibility augmenting agents and an active agent. The slurry is introduced into the drying chamber 4 as heat 3 is applied. As the atomized slurry dries, the powder collected in the collection system 5 includes agglomerated particles of the active agent.

In accordance with another embodiment of the present invention, the slurry is a mixture of wetted active agent, microcrystalline cellulose in the form of a wet cake (hydrocellulose) and at least one compressibility augmenting agent, e.g., colloidal silicon dioxide, wherein the slurry is introduced into the drying chamber 4 as heat 3 is applied. As the atomized fluid material dries, the powder collected in the collection system 5 includes agglomerated particles of the active agent.

In certain other embodiments, the compressibility augmenting agent may be combined with the active agent/microcrystalline cellulose slurry in the dryer.

In yet another embodiment, additional compressibility augmenting agents or pre-manufactured agglomerated particles, e.g., ProSolv® HD, may be combined with the active agent slurry in the dryer. In other embodiments, additional compressibility augmenting agents, pre-manufactured agglomerated particles consisting of microcrystalline cellulose and compressibility augmenting agent or additional pharmaceutically acceptable excipients may be mixed together with the active agent agglomerated particles prior to incorporation into a solid dosage form utilizing art know techniques.

It is hypothesized that the granulation-reduced microcrystalline cellulose compactibility is caused at least in significant part by increasing intraparticle and/or interparticle hydrogen bonding. For purposes of the present invention, this phenomenon is termed “quasi-hornification” since, unlike hornification of cellulose fibers described in the literature elsewhere, quasi-hornification of microcrystalline cellulose has not been observed by us to reduce the ability of microcrystalline cellulose to absorb water vapor. Furthermore, quasi-hornified microcrystalline cellulose was found to be fully reversible, unlike the hornification which occurs when cellulose is wetted. Microcalorimetry indicates that during adsorption of water vapor by granulated microcrystalline cellulose, the extent of intraparticle bond disruption is greater than occurring during water vapor adsorption by ungranulated microcrystalline cellulose. This provides evidence to support the theory that granulation results in increased intraparticle hydrogen bonding, some of which is reversible on adsorption of water vapor.

As noted above, by slurry, it is meant that the material (e.g., the active agent alone or the mixture of active agent, microcrystalline cellulose and compressibility augmenting agent) is sufficiently wetted to be suitable for subsequent spray drying. For example, the fluid material may be in a solution, a suspension, a slurry, or an emulsion. Moreover, the slurry may include one or more of a variety of solvents, including water, alcohol, ethanol, and the like. Hydro-alcohol solvents may also be used.

Preparation of Active Agent Agglomerated Particles

In certain embodiments, the active agglomerated particles are formed by a) preparing a slurry of pre-manufactured agglomerated particles consisting of microcrystalline cellulose and one or more compressibility augmenting agents, and an active agent; and b) drying the slurry to form active agent agglomerated particles.

In certain embodiments, active agglomerated particles are formed by: a) preparing an aqueous slurry of microcrystalline cellulose, a compressibility augmenting agent and other, optional, pharmaceutically acceptable excipients; b) drying the mixture of ingredients prepared in step a) in a manner which inhibits quasi-hornification of the microcrystalline cellulose to obtain agglomerate particles; c) preparing a slurry containing the agglomerated particles obtained in step b) together with a suitable amount of an active agent, and other, optional, pharmaceutically acceptable excipients; and; d) drying the slurry to form active agent agglomerated particles.

The slurry should be thoroughly agitated during preparation with a suitable device (like an electric lab stirrer). The desired temperature for heating the slurry will vary depending on the solubility of the active agent and the temperature that the active agent begins to degrade. For example, Glucosamine HCl has a temperature dependent solubility in water that is greater than about 60° C.

In certain embodiments, the active agent may be dispersed in a solvent prior to combining the active agent into the slurry. In certain other embodiments, instead of completely dispersing the active agent into a particular form of slurry, it is preferable to prepare a blend of active agent slurries, such as a slurry-solution blend that is suitable for coprocessing the active agent with the microcrystalline cellulose/compressibility augmenting agent slurry.

Regardless of the sequence of adding the active agent to the slurry, the slurry should be mixed until a homogenous mixture is achieved. Preferably, the active agent is slowly added to the slurry or visa versa as adding the active too quickly may cause it to clump up on top of the slurry and not disperse thoroughly. Once active agent and slurry are mixed together, the slurry can then be fed into a drying chamber so that the slurry is atomized.

The compressibility augmenting agents for use in the present invention should be capable of (i) physically restricting the proximity of the interface between adjacent cellulose surfaces; (ii) inhibiting interactions between adjacent cellulose surfaces, for example, via the creation of a hydrophobic boundary at cellulose surfaces; or (iii) accomplishing both (i) and (ii) above. Suitable compressibility augmenting agents will preferably have an HLB value of at least 10, preferably at least about 15, and more preferably from about 15 to about 40 or greater. To date, compressibility augmenting agents which have shown the greatest effect have had relatively high HLB values, and therefore an HLB value from about 30 to about 40 or greater is most preferred. Agents which exhibit these properties include certain surfactants such as sodium lauryl sulfate and polysorbate 40, and highly polar compounds, including pharmaceutically acceptable dyes such as congo red.

In accordance with still other embodiments of the present invention, additional compressibility augmenting agents may include pharmaceutically (or nutraceutically) acceptable metal oxides such as colloidal titanium oxide, or colloidal carbon black.

In one preferred embodiment of the invention, the compressibility augmenting agent which provides a physical barrier between adjacent cellulose surfaces is a silicon dioxide.

Silicon dioxide is obtained by precipitating dissolved silica in sodium silicate solution. When obtained by the addition of sodium silicate to a mineral acid, the product is termed silica gel. When obtained by the destabilization of a solution of sodium silicate in such a manner as to yield very fine particles, the product is termed precipitated silica. Silicon dioxide is insoluble in water. Prior to the present invention, silicon dioxide, and in particular colloidal silicon dioxide, was used mainly as a glidant and anti-adherent in tableting processes and encapsulation, promoting the flowability of the granulation. The amount of silicon dioxide included in such tablets for those applications is very limited, 0.1-0.5% by weight. Handbook of Pharmaceutical Excipients, .COPYRGT.1986 American Pharmaceutical Association, page 255. This is due in part to the fact that increasing the amount of silicon dioxide in the mixture to be tableted causes the mixture to flow too well, causing a phenomena known to those skilled in the tableting art as “flooding”. If the mixture flows too well, a varying tablet weight with uneven content uniformity can result.

Those skilled in the art will appreciate that the name and/or method of preparation of the silicon dioxide utilized in the present invention is not determinative of the usefulness of the product. Rather, as previously mentioned, it has been surprisingly discovered that it is the physical characteristics of the silicon dioxide that are critical. In particular, it has been discovered that silicon dioxide having a relatively large particle size (and correspondingly small surface area), such as silica gel, is not useful in the preparation of the improved microcrystalline cellulose products of the invention. The appended claims are deemed to encompass all forms of silicon dioxide having an average primary particle size from about 1 nm to about 100 μm, and/or a surface area from about 10 m²/g to about 500 m²/g.

In the context of the present invention, silicified MCC (agglomerated particles of microcrystalline cellulose and silicon dioxide) is a particulate agglomerate of coprocessed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (μm), based on average primary particle size. By “intimate association”, it is meant that the silicon dioxide has in some manner been integrated with the microcrystalline cellulose particles, e.g., via a partial coating of the microcrystalline particles, as opposed to a chemical interaction of the two ingredients. The term “intimate association” is therefore deemed for purposes of the present description as being synonymous with “integrated” or “united”. The coprocessed particles are not necessarily uniform or homogeneous. Rather, under magnification, e.g., scanning electron microscope at 500 times, the silicon dioxide at the preferred percent inclusion appears to be an “edge-coating”. Preferably, the silicon dioxide comprises from about 0.5% to about 10% of the silicified MCC, and most preferably from about 1.25% to about 5% by weight relative to the microcrystalline cellulose. Moreover, the silicon dioxide preferably has a particle size from about 5 nm to about 40 μm, and most preferably from about 5 nm to about 50 μm. Moreover, the silicon dioxide preferably has a surface area from about 10 m2 g to about 500 m2/g, preferably from about 50 m2/g to about 500 m2/g, and more preferably from about 175 m²/g to about 350 m²/g. Silicified MCC, and methods for its manufacture, are described in U.S. Pat. No. 5,585,115, the entire disclosure of which is hereby incorporated by reference. Silicified microcrystalline cellulose is commercially available from JRS Pharma, Inc., under the trademark Prosolv®. Prosolv® is available in a number of grades, including, for example, Prosolv® SMCC 50, Prosolv® SMCC 90, and Prosolv® HD, each of which contains 2% colloidal silicon dioxide, by weight relative to the microcrystalline cellulose.

Colloidal silicon dioxide is a submicron fumed silica prepared by the vapor-phase hydrolysis (e.g., at 1110° C., or 1800° C.) of a silicon compound, such as silicon tetrachloride. The product itself is a submicron, fluffy, light, loose, bluish-white, odorless and tasteless amorphous powder which is commercially available from a number of sources, including Cabot Corporation (under the tradename Cab-O-Sil); Degussa, Inc. (under the tradename Aerosil); E. I. DuPont & Co.; and W. R. Grace & Co. Colloidal silicon dioxide is also known as colloidal silica, fumed silica, light anhydrous silicic acid, silicic anhydride, and silicon dioxide fumed, among others. A variety of commercial grades of colloidal silicon dioxide are produced by varying the manufacturing process. These modifications do not affect the silica content, specific gravity, refractive index, color or amorphous form. However, these modifications are known to change the particle size, surface areas, and bulk densities of the colloidal silicon dioxide products.

The surface area of the preferred class of silicon dioxides utilized in the invention ranges from about 50 m²/gm to about 500 m²/gm. The average primary particle diameter of the preferred class of silicon dioxides utilized in the invention ranges from about 5 nm to about 50 nm. However, in commercial colloidal silicon dioxide products, these particles are agglomerated or aggregated to varying extents. The bulk density of the preferred class of silicon dioxides utilized in the invention ranges from about 20 g/l to about 100 μl.

Commercially available colloidal silicon dioxide products have, for example, a BET surface area ranging from about 50+/−15 m²/gm (Aerosil OX50) to about 400+/−20 (Cab-O-Sil S-17) or 390+/−40 m²/gm (Cab-O-Sil EH-5). Commercially available particle sizes range from a nominal particle diameter of 7 nm (e.g., Cab-O-Sil S-17 or Cab-O-Sil EH-5) to an average primary particle size of 40 nm (Aerosil OX50). The density of these products range from 72.0+−8 μl (Cab-O-Sil S-17) to 36.8 g/l (e.g., Cab-O-Sil M-5). The pH of these products at 4% aqueous dispersion ranges from pH 3.5-4.5. These commercially available products are described for exemplification purposes of acceptable properties of the preferred class of silicon dioxides only, and this description is not meant to limit the scope of the invention in any manner whatsoever.

Another type of colloidal silicon dioxide is surface treated silica, including, for example, hydrophobically modified silica and hydrophilically modified silica. An example of a commercially available hydrophobically modified silica that may be used as the colloidal silicon dioxide in the embodiments described herein is AEROSIL® R 972, manufactured by Degussa AG.

In certain embodiments, colloidal silicon dioxide is preferred.

In addition to the active agent being added to the slurry of pre-manufactured agglomerated particles, in certain other embodiments, a surfactant such as sodium lauryl sulfate (SLS) may be added to the slurry together with the active agent. In certain other embodiments, a surfactant active agent slurry may be prepared prior to addition to the slurry of pre-manufactured agglomerated particles. In either process, the surfactant is incorporated into the slurry prior to drying.

### Dryer Set-Up

During the preparation of the slurry, the spray dryer is started up and allowed to run until the desired inlet and outlet temperatures are reached. One skilled in the art would understand that the desired inlet and outlet temperatures may vary depending on the equipment being utilized for drying and the stability of the active agent and additional ingredients. However, the desired inlet and outlet temperatures should be set such that charring of the materials is avoided and the desired moisture content of the collected material is achieved. Once the desired inlet temperature is achieved the oil pump for the atomizer is started, with the oil pump started the atomizer can be run at about 50 Hz (this may also vary depending on the equipment utilized). Once the desired dryer outlet temperature is achieved the feed pump can be started and the dryer run on water. Running water into the dryer helps achieve the desired inlet and outlet temperatures and aids in maintaining those temperatures until actual drying of the slurry is to take place. Periodic adjustment to the water flow may be needed to maintain temperature. For example, when co-processing glucosamine, the desired inlet temperature should be about 240° C., and desired outlet temperature should be about 99° C.

With the temperature stabilized, drying of the slurry may commence. The slurry material is pumped into the atomizer and the feed rate adjusted to obtain the desired outlet temperature. Once the dryer has achieved a steady state at the desired temperatures, periodic samples are taken and measured on a particle size analyzer to ensure that the desired particle size is achieved, e.g., about 65 μM. Particle size may vary depending on the active agent and amount used and the amount of pre-manufactured agglomerated particles used to form the slurry. Suitable particle sizes for the agglomerated particles may range from about 10 μm to about 400 μm, about 10 μm to about 300 μm; preferably from 30 μm to about 125 μm; and more preferably about 65 μm to about 100 μm. The particle size of the active agent agglomerated particles may also be affected by the inlet and outlet temperatures and the feed rate of active agent/silicified MCC fluid material into the dryer. The atomizer speed may also be changed to achieve the desired particle size. Once the material meets the particle size specification then it is acceptable to begin collecting product. The coprocessing run should be continued until all the slurry has been used. Once all the slurry has been used, the collection vessel should be changed so that good product is segregated from dryer shutdown material. The startup and shutdown coprocessed materials should be saved until they are deemed of no use.

### Active Agents

The active agent(s) which may be used in accordance with the embodiments described above include systemically active therapeutic agents, locally active therapeutic agents, disinfecting agents, chemical impregnants, cleansing agents, deodorants, fragrances, dyes, animal repellents, insect repellents, fertilizing agents, pesticides, herbicides, fungicides, plant growth stimulants, and the like.

A wide variety of therapeutically active agents can be used in conjunction with the present invention. The therapeutically active agents (e.g. pharmaceutical agents) include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, piroxicam and sulindac), anti-emetics (e.g., metoclopramide), anti-epileptics (e.g., phenyloin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluazide), anti-hypotensives (e.g., propranolol, clonidine), antihypertensives (e.g., clonidine, methyldopa), bronchodilators (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants, laxatives, vitamins, stimulants (including appetite suppressants such as phenylpropanolamine).

In certain embodiments, the active agent is ramipril.

The above list is not meant to be exclusive and includes any pharmaceutically acceptable salt, derivative or mixtures thereof.

In one embodiment of the present invention, the active agent is glucosamine and its pharmaceutically acceptable salts and esters, including, for example, glucosamine, glucosamine HCL, glucosamine SO4Na, and glucosamine SO4K. In another embodiment, the active agent is chondroitin and its pharmaceutically acceptable salts and esters, including chondroitin sulfate. In still other embodiments, the active agent includes both glucosamine and its pharmaceutically acceptable salts and esters and chondroitin and its pharmaceutically acceptable salts and esters.

A wide variety of locally active agents can be used in conjunction with the embodiments described herein, and include both water soluble and water insoluble agents. The locally active agent(s) is intended to exert its effect in the environment of use, e.g., the oral cavity, although in some instances the active agent may also have systemic activity via absorption into the blood via the surrounding mucosa.

The locally active agent(s) include antifungal agents (e.g., amphotericin B, clotrimazole, nystatin, ketoconazole, miconazol, etc.), antibiotic agents (penicillins, cephalosporins, erythromycin, tetracycline, aminoglycosides, etc.), antiviral agents (e.g., acyclovir, idoxuridine, etc.), breath fresheners (e.g. chlorophyll), antitussive agents (e.g., dextromethorphan hydrochloride), anti-cariogenic compounds (e.g., metallic salts of fluoride, sodium monofluorophosphate, stannous fluoride, amine fluorides), analgesic agents (e.g., methylsalicylate, salicylic acid, etc.), local anesthetics (e.g., benzocaine), oral antiseptics (e.g., chlorhexidine and salts thereof, hexylresorcinol, dequalinium chloride, cetylpyridinium chloride), anti-inflammatory agents (e.g., dexamethasone, betamethasone, prednisone, prednisolone, triamcinolone, hydrocortisone, etc.), hormonal agents (oestriol), antiplaque agents (e.g., chlorhexidine and salts thereof, octenidine, and mixtures of thymol, menthol, methysalicylate, eucalyptol), acidity reducing agents (e.g., buffering agents such as potassium phosphate dibasic, calcium carbonate, sodium bicarbonate, sodium and potassium hydroxide, etc.), and tooth desensitizers (e.g., potassium nitrate). This list is not meant to be exclusive. The solid formulations of the invention may also include other locally active agents, such as flavorants and sweeteners. Generally any flavoring or food additive such as those described in Chemicals Used in Food Processing, pub 1274 by the National Academy of Sciences, pages 63-258 may be used. Generally, the final product may include from about 0.1% to about 5% by weight flavorant.

In accordance with one embodiment of the present invention, the active agent is a liquid herbal extract. As noted above, the term “liquid” as used herein means that the herbal extract is sufficiently wetted to be atomized in a spray dryer. Preferably, the herbal extract is selected from the group consisting of: Alfalfa Leaf, Alfalfa Juice, Aloee-emodin, Andrographolide, Angelica Root, Astragalus Root, Bilberry, Black Cohosh Root, Black Walnut Leaf, Blue Cohosh Root, Burdock Root, Cascara Bark, Cats Claw Bark, Catnip Leaf, Cayenne, Chamomile Flowers, Chaste Tree Berries, Chickweed, Chinese Red Sage Root, Cranberry, Chrysophanol, Comfrey Leaf, Cramp Bark, Damiana Leaf, Dandelion Root CO, Devil's Claw Root, Diosgenin, Dong Quai Root, Dong Quai, Echinacea, Echinacea Angustifolia Root, Echinacea Purpurea Herb Root and Echinacea Angust./Purpurea Blend CO, Echinacea Angust./Goldenseal Blend, Eleuthero (Siberian) Ginseng Root, Emodin, Eyebright Herb, Fenugreek, Feverfew Herb CO, Fo-Ti Root, Fo-Ti, Garcinia Cambogia, Gentian Root, Ginger, Ginko Biloba Ginger Root, Ginseng, Ginko Leaf, Ginseng Root, Goldenseal Root, Gotu Kola Herb, Grape Seed, Grape Skin, Green Tea, Green Tea, Decaf, Guarana Seeds, Gynostemma Pentaphyllum, Hawthorn Berries, Hawthorn Leaf, Hesperdin, Hops Flowers, Horehound Herb, Horse Chestnut, Horsetail, Hyssop Leaf, Huperzine A, Juniper Berries, Kava Kava Root, Kola Nut, Lavender Flowers, Lemon Balm, Licorice Root, Lobelia Herb, Lomatium, Marshmallow Root, Milk Thistle Seed, Milk Thistle, Mullein Leaf, Myrrh, Naringin, Neohesperidin, Nettle Leaf, Olive Leaf, Oregon Grape Root, Papain, Parsley Leaf & Root, Passion Flower, Pau D'Arco Bark, Pennyroyal, Peppermint Leaf, Physcion, Polystictus Versicolor Mushroom, Quercetin, Red Clover Blossoms, Red Clover, Red Raspberry Leaf, Red Yeast Rice, Reishi Mushrooms, Rhein, Rhubarb Root, Rosemary Leaf, Rutin, Sarsaparilla Root, Saw Palmetto, Saw Palmetto Berry, Schisandra Berries, Schisandra, Scullcap Herb, Shavegrass Herb, Sheep Sorrel, Shepard's Purse Herb, Shitake Mushroom, Slippery Elm Bark, Sown Orange, Soybean, Stevia Rebaudiana, St. John's Wort, Tetrandrine, Turmeric, Usnea Lichen, Uva Ursi, Uva Ursi Leaf, Valerian Root, White Willow Bark, Wild Yam Root, Yellow Dock Root, Yohimbe Bark, Yucca Root, and combinations thereof. Most preferably, the herbal extract is selected from the group consisting of St. John's Wort, Artichoke Leaves, and Ginseng.

In accordance with certain embodiments of the present invention, the active agent is hygroscopic. Examples of hygroscopic active agents include many herbal extracts, including St. John's Wort, Artichoke Leaves, and Ginseng.

### Advantages of API Co-Processing

The agglomerated particles in accordance with the embodiments of the present invention described above provide a number of advantages. Specifically, the agglomerated particles provide superior flow characteristics to prior art compositions. As one of ordinary skill in the art will appreciate, the superior flow characteristics provided by the embodiments of the present invention allow faster and more efficient processing for tablets, capsules, and other dosage forms.

The agglomerated particles in accordance with the embodiments of the present invention also provide superior compaction characteristics to prior art compositions. As one of ordinary skill in the art will appreciate, the superior compaction characteristics provided by the embodiments of the present invention allow faster and more efficient processing for tablets, and, moreover, allow a larger percentage of active agent to be included in each tablet.

In addition, the agglomerated particles in accordance with the embodiments of the present invention exhibit superior content uniformity when tableted than agglomerated particles that are formed by a wet granulation of silicified MCC and an active agent. This is particularly useful when tableting low dose formulations because such formulations are particularly prone to content uniformity problems. Thus, the agglomerated particles in accordance with certain embodiments of the present invention are particularly advantageous with respect to tablets including 100 mg or less active agent in tablets having a total tablet weight between 200 mg and 800 mg. In certain embodiments, the tablets include 50 mg or less active agent in tablets having a total tablet weight of between 200 mg and 800 mg. In other embodiments, the tablets include 10 mg or less active agent in tablets having a total tablet weight of between 50 mg and 800 mg. In still other embodiments, the tablets include 1 mg or less active agent in tablets having a total tablet weight of between 10 mg and 800 mg. In still other embodiments, the tablets include no more than about 20% by weight active agent, preferably no more than about 10% by weight active agent, and most preferably no more than about 1% by weight active agent.

Prior to being incorporated into a solid dosage form, the agglomerated particles may be combined with additional pharmaceutically acceptable excipients such as those described in the Handbook of Pharmaceutical Excipients, American Pharmaceutical Association, 4th Edition (2003), the disclosure of which is hereby incorporated by reference. Examples of suitable pharmaceutically acceptable excipients include, but are not limited to, binders, diluents, disintegrators, lubricants, preserving agents, fillers, surfactants and wetting agents, emulsifying agents, suspending agents, sweetening agents, flavoring agents, perfuming agents, and dispensing agents, etc.

Binders suitable for use in the present invention include, but are not limited to, acacia, alginic acid, tragacanth, sucrose, gelatin, glucose, starch, cellulose derivatives (e.g., methyl cellulose, sodium carboxymethylcellulose), hydroxypropylmethylcellulose, ethyl cellulose, polyvinylpyrrolidone (PVP), sodium alginate, polyethyleneglycols, guar gum, polysaccharide acids, bentonites, the mixtures thereof, etc.

Diluents suitable for use in the present invention include, but are not limited to, pharmaceutically accepted hydrogels such as alginate, chitosan, methylmethacrylates, a monosaccharide, a disaccharide, a polyhydric alcohol, a cellulose or derivatives thereof (microcrystalline cellulose, hydroxypropyl cellulose, hydroxypropyl methyl cellulose, carboxymethylcellulose, ethylcellulose), agarose and Povidone™, kaolin, magnesium stearate, starch, lactose, sucrose, density-controlling agents such as barium sulfate and oils, dissolution enhancers such as aspartic acid, citric acid, glutamic acid, tartartic acid, sodium bicarbonate, sodium carbonate, sodium phosphate, glycine, tricine and TRIS. In certain embodiments the diluent may be an augmented microcrystalline cellulose as disclosed in U.S. Pat. No. 5,585,115, the disclosure of which is hereby incorporated by reference.

In certain embodiments, part or all of the diluent may comprise a pre-manufactured direct compression diluent. Suitable pre-manufactured direct compression diluents include, but are not limited to, Emcocel® (microcrystalline cellulose, N.F.), Emdex® (dextrates, N.F.), and Tab-Fine® (a number of direct-compression sugars including sucrose, fructose, and dextrose), all of which are commercially available from JRS Pharma LP, Patterson, N.Y.). Other direct compression diluents include anhydrous lactose (Lactose N.F., anhydrous direct tableting) from Sheffield Chemical, Union, N.J. 07083; Elcema® G-250 (Powdered cellulose, N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltrodextrin) from Grain Processing Corp., Muscatine, Iowa 52761; Neosorb 60® (Sorbitol, N.F., direct-compression) from Roquette Corp., 645 5th Ave., New York, N.Y. 10022; Nu-Tab® (Compressible sugar, N.F.) from Ingredient Technology, Inc., Pennsauken, N.J. 08110; Poly plasdone XL® (Crospovidone, N.F., cross-linked polyvinylpyrrolidone) from GAF Corp., New York, N.Y. 10020; Primojel® (Sodium starch glycolate, N.F., carboxymethyl starch) from Generichem Corp., Little Falls, N.J. 07424; Solka Floc® (Cellulose floc) from International Fiber Corp., N.Y., Spray-dried Lactose® (Lactose N.F., spray dried) from Foremost Whey Products, Baraboo, Wis. 53913 and DMV Corp., Vehgel, Holland; and Sta-Rx 1500® (Starch 1500) (Pregelatinized starch, N.F., compressible) from Colorcon, Inc., West Point, Pa. 19486.

Disintegrants suitable for use in the present invention may include, but are not limited to, starches, starch derivatives (e.g., low substituted carboxymethylcellulose starches, hydroxypropyl starch, etc.), clays (e.g., Veegum HV and Bentonite, etc.), celluloses (e.g., purified cellulose, methylcellulose, sodium carboxymethylcellulose, carboxymethylcellulose, microcrystalline cellulose, silicified microcrystalline cellulose, etc.), alginates (e.g., alginic acid, sodium alginate, etc.), pregelatinized corn starches, gums (e.g., agar, guar, karaya, traganth, etc.), surfactants, resins, effervescent mixtures, polyvinylpyrolidone, complex silicates, etc. The amount of disintegrant incorporated into each controlled release component may vary in a range from about 0.1% to about 99% of the formulation by weight.

Lubricants suitable for use in the present invention include, but are not limited to, a metallic stearate (e.g., magnesium stearate, calcium stearate, sodium stearate, etc.), stearic acid, talc, waxes, surfactants (e.g., sodium lauryl sulfate, magnesium lauryl sulfate, etc.), starch, silica, high molecular weight polyethylene glycols, etc. When the lubricant utilized is a metallic stearate, a metal concentration of the formulation/composition is more than 1 ppm. The lubricant may comprise, for example, magnesium stearate in any amount of about 0.5-3% by weight of the solid dosage form.

Surfactants or wetting agents suitable for use in the present invention include, but are not limited to, anionic surfactants, cationic surfactants, amphoteric (amphipathic/amphophilic) surfactants, and non-ionic surfactants. Examples of suitable surfactant or wetting agents include, inter alia, alkali metal chlorides, magnesium chloride, calcium chloride, organic acids such as citric, succinic, fumaric, malic, maleic, glutaric, lactic and the like, alkali metal sulfates such as sodium sulfate, alkali metal alkyl sulfates wherein the alkyl group is from 1 to 14 carbon atoms, such as sodium methyl sulfate, sodium lauryl sulfate and the like as well as dioctyl sodium sulfosuccinate, dihydrogen sodium phosphate, monohydrogen sodium phosphate, disodium hydrogen phosphate, sodium chloride, sodium fluoride and mixtures thereof, polyethyleneglycols as esters or ethers, polyethoxylated castor oil, polyethoxylated hydrogenated castor oil, polyethoxylated fatty acid from castor oil or polyethoxylated fatty acid from castor oil or polyethoxylated fatty acid from hydrogenated castor oil. Commercially available wetting agents which can be used are known under trade names Cremophor, Myrj, Polyoxyl 40 stearate, Emerest 2675, Lipal 395 and PEG 3350.

The complete mixture, in an amount sufficient to make a uniform batch of tablets, may then subjected to tableting in a conventional production scale tableting machine at normal compression pressures for that machine, e.g., about 1500-10,000 lbs/sq in. The mixture should not be compressed to such a degree that there is subsequent difficulty in its hydration when exposed to gastric fluid.

The average tablet size for round tablets is preferably about 50 mg to 500 mg and for capsule-shaped tablets about 200 mg to 2000 mg. However, other formulations prepared in accordance with the present invention may be suitably shaped for other uses or locations, such as other body cavities, e.g., periodontal pockets, surgical wounds, vaginally. It is contemplated that for certain uses, e.g., antacid tablets, vaginal tablets and possibly implants, that the tablet will be larger.

In certain embodiments of the invention, the tablet is coated with a sufficient amount of a hydrophobic polymer to render the formulation capable of providing a release of the medicament such that a 12 or 24 hour formulation is obtained. In other embodiments of the present invention, the tablet coating may comprise an enteric coating material in addition to or instead or the hydrophobic polymer coating. Examples of suitable enteric polymers include cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit™ L 100-555.

In further embodiments, the dosage form may be coated with a hydrophilic coating in addition to or instead of the above-mentioned coatings. An example of a suitable material which may be used for such a hydrophilic coating is hydroxypropylmethylcellulose (e.g., Opadry®, commercially available from Colorcon, West Point, Pa.).

The coatings may be applied in any pharmaceutically acceptable manner known to those skilled in the art. For example, in one embodiment, the coating is applied via a fluidized bed or in a coating pan. For example, the coated tablets may be dried, e.g., at about 60°-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

The coatings which may be optionally applied to the compressed solid dosage form of the invention may comprise from about 0.5% to about 30% by weight of the final solid dosage form.

In additional embodiments of the present invention, a support platform is applied to the tablets manufactured in accordance with the present invention. Suitable support platforms are well known to those skilled in the art. An example of suitable support platforms is set forth, e.g., in U.S. Pat. No. 4,839,177, hereby incorporated by reference. In that patent, the support platform partially coats the tablet, and consists of a polymeric material insoluble in aqueous liquids. The support platform may, for example, be designed to maintain its impermeability characteristics during the transfer of the therapeutically active medicament. The support platform may be applied to the tablets, e.g., via compression coating onto part of the tablet surface, by spray coating the polymeric materials comprising the support platform onto all or part of the tablet surface, or by immersing the tablets in a solution of the polymeric materials.

The support platform may have a thickness of, e.g., about 2 mm if applied by compression, and about 10 μm if applied via spray-coating or immersion-coating. Generally, in embodiments of the invention wherein a hydrophobic polymer or enteric coating is applied to the tablets, the tablets are coated to a weight gain from about 1% to about 20%, and in certain embodiments preferably from about 5% to about 10%.

Materials useful in the hydrophobic coatings and support platforms of the present invention include derivatives of acrylic acid (such as esters of acrylic acid, methacrylic acid, and copolymers thereof) celluloses and derivatives thereof (such as ethylcellulose), polyvinylalcohols, and the like.

In certain embodiments of the present invention, an additional dose of the active agent may be included in either the hydrophobic or enteric coating, or in an additional overcoating coated on the outer surface of the tablet core (without the hydrophobic or enteric coating) or as a second coating layer coated on the surface of the base coating comprising the hydrophobic or enteric coating material. This may be desired when, for example, a loading dose of a therapeutically active agent is needed to provide therapeutically effective blood levels of the active agent when the formulation is first exposed to gastric fluid. The loading dose of active agent included in the coating layer may be, e.g., from about 10% to about 40% of the total amount of medicament included in the formulation.

The tablets of the present invention may also contain effective amounts of coloring agents, (e.g., titanium dioxide, F.D. & C. and D. & C. dyes; see the Kirk-Othmer Encyclopedia of Chemical Technology, Vol. 5, pp. 857-884, hereby incorporated by reference), stabilizers, binders, odor controlling agents, and preservatives.

Alternatively, the agglomerated particles of active agent/silicified MCC (with or without sodium lauryl sulfate (“SLS”), can be utilized in other applications wherein it is not compressed. For example, the agglomerated particles can be filled into capsules. The agglomerated particles can further be molded into shapes other than those typically associated with tablets. For example, the agglomerated particles can be molded to “fit” into a particular area in an environment of use (e.g., an implant). All such uses would be contemplated by those skilled in the art and are deemed to be encompassed within the scope of the appended claims.

### DETAILED DESCRIPTION OF THE PREFERRED EMBODIMENTS

### Examples

### Examples 1

### Piroxicam Co-Processed Agglomerated Particles

### Example 1

### Piroxicam:ProSolv® (75:25)

In this example a Piroxicam/ProSolv® slurry was prepared in a ratio of 75:25 with the expectation of obtaining a particle size about 65 μm.

| Materials: | Piroxicam | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | .97 kg |
| | Piroxicam % | 75% |
| | Required Piroxicam | .7275 kg |
| | ProSolv % | 25% |
| | Required ProSolv | .2425 kg |
| | Solids Content | 40% |
| | Inlet Temp | 240° C. |
| | Outlet Temp | 99° C. |
| | Feed Rate | 9 |
| | Atomizer Frequency | 50 Hz |
| | Gas Pressure | 6.8″ H2O |

### CONCLUSION

During the initial mixing of the slurry an additional 2 kg of water had to be added in order to slurry the Piroxicam. The additional 2 kg of water lowered the solids content to 19%. The run was completed and the yield was low, and at first appearance the material seemed very fine.

### Example 2

### Piroxicam:ProSolv® (60:40)

The original Piroxicam trial utilized a 75/25 ratio of Piroxicam to ProSolv that produced a very fine product. In this example a Piroxicam/ProSolv® slurry was prepared in a ratio of 60:40 with a lowered solids content (19%) in the hope that the changed configurations will increase yield as well as produce a particle size around 65 μm.

| Materials: | Piroxicam | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | 2.5 kg |
| | Piroxicam % | 60% |
| | Required Piroxicam | 1.5 kg |
| | ProSolv % | 40% |
| | Required ProSolv | 1 kg |
| | Solids Content | 19.05% |
| | Inlet Temp | 240° C. |
| | Outlet Temp | 99° C. |
| | Feed Rate | 9 |
| | Atomizer Frequency | 50 Hz/30 Hz |
| | Gas Pressure | 6.8″ H2O |

### CONCLUSION

The co-processing trial has been completed and two different samples were taken during the run, a 50 Hz sample and a 30 Hz sample. The lowered solids content increased the yield of the run.

### Example 3

### Piroxicam:ProSolv® (40:60)

In this example a Piroxicam/ProSolv® slurry was prepared in a ratio of 40:60 with a lowered solids content (12%) to make it easier to slurry and the atomizer was run slower in the hope of achieving larger particle size material.

| Materials: | Piroxicam | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | 2.5 kg |
| | Piroxicam % | 40% |
| | Required Piroxicam | 1 kg |
| | ProSolv % | 60% |
| | Required ProSolv | 1.5 kg |
| | Solids Content | 12% |
| | Inlet Temp | 240° C. |
| | Outlet Temp | 99° C. |
| | Feed Rate | 11 |
| | Atomizer Freq | 25/15 Hz |
| | Gas Pressure | 6.8″ H2O |
| | Particle Size | 25 Hz-23 pem/15 Hz-14 pem |

### CONCLUSION

The material was bagged and collected, particle size analysis was done and there was little difference in the particle size of the newly produced material versus the first coprocessed Piroxicam.

### Example 4

### Piroxicam:ProSolv® (25:75)

In this example a Piroxicam/ProSolv® slurry was prepared in a ratio of 25:75 and the solids content was lowered to 7.5% with the hope of increasing the particle size to make the end powder more workable.

| Materials: | Piroxicam | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | 2.5 kg |
| | Piroxicam % | 25% |
| | Required Piroxicam | .625 kg |
| | ProSolv % | 75% |
| | Required ProSolv | 1.875 kg |
| | Solids Content | 7.50% |
| | Inlet Temp | 240° C. |
| | Outlet Temp | 99° C. |
| | Feed Rate | 12 |
| | Atomizer Freq | 25 Hz |
| | Gas Pressure | 6.9H2O |
| | Particle Size | 48 μm |

### CONCLUSION

The material was bagged and collected. Particle size analysis was done and the particle size for this batch was 48 μm as compared to 15-25 μm of previous runs.

### Example 5

### Piroxicam:ProSolv®

A new batch of coprocessed Piroxicam has been made. The particle size on the new batch was about 50 μm. Tablets were made and tested to determine if the increased particle size effected compaction.

| Materials: | Piroxicam | |
|---|---|---|
| | ProSolv 50 (98% microcrystalline cellulose:2% | |
| | colloidal silicon dioxide) | |
| Equipment: | Korsch PH 106 Rotary Tablet Press | |
| | Erweka TBH-30 | |
| | Balance #0274 | |
| | Hygrometer #0526 | |
| Methods: | Press Speed | 50 rpm |
| | Punch Size | ⅜″ round flat face |
| | Target Weight | 250 mg (#5) |
| | Compaction Force | 3, 6, 9, 12, 15kN |

1) Take 10 tablets and weigh them for the average mass.
2) Take the same 10 tablets and put them on the hardness tester to measure hardness, thickness and diameter.
3) Save 10-15 tablets for further testing.
4) Repeat for each blend.

Tablet compaction force for the tablets made in this example are set forth in Table I below.

TABLE I

| Avg | Avg | |
|---|---|---|
| Mass | Force | Std. |
| (mg) | (kN) | Dev |
| 245.07 | 3.42 | .11 |
| 249.31 | 6.35 | .45 |
| 246.91 | 8.79 | .45 |
| 253.45 | 13.01 | .45 |
| 248.62 | 15.55 | .41 |

Temp: 19.1° C.
RH: 36%

Hardness, thickness, tensile strength and diameter values for 10 tablet samples prepared with a compression force of 3.42 kN, 13.01 kN, 6.35 kN, 15.55 kN and 8.79 kN are set forth in Tables II-VI respectively, provided below.

TABLE II
Compression Force
3.42 kN

| | Hardness | Thickness | Diameter | Tensile |
|---|---|---|---|---|
| Tablet # | (N) | (mm) | (mm) | Strength (MPa) |
| 1 | 73 | 2.98 | 9.44 | 1.652018 |
| 2 | 72 | 2.99 | 9.45 | 1.62222 |
| 3 | 83 | 3.02 | 9.44 | 1.853443 |
| 4 | 65 | 2.98 | 9.43 | 1.472535 |
| 5 | 79 | 3.01 | 9.43 | 1.754373 |
| 6 | 64 | 2.98 | 9.43 | 1.44900 |
| 7 | 62 | 3.03 | 9.43 | 1.381394 |
| 8 | 66 | 3.01 | 9.43 | 1.480287 |
| 9 | 60 | 3 | 9.43 | 1.552731 |
| 10 | 70 | 3 | 9.43 | 1.5795235 |
| Average | 70.3 | 3.003 | 9.434 | 1.579411 |
| Std. dev. | 6.700746 | 0.0216282 | 0.006992 | 0.145593 |

TABLE III
Compression Force
13.01 kN

| | Hardness | Thickness | Diameter | Tensile |
|---|---|---|---|---|
| Tablet # | (N) | (mm) | (mm) | Strength (MPa) |
| 1 | 235 | 2.62 | 9.43 | 6.055291 |
| 2 | 209 | 2.59 | 9.43 | 5.447722 |
| 3 | 253 | 2.66 | 9.42 | 6.427885 |
| 4 | 237 | 2.61 | 9.42 | 6.136731 |
| 5 | 240 | 2.61 | 9.42 | 6.214411 |
| 6 | 229 | 2.58 | 9.42 | 5.998532 |
| 7 | 262 | 2.66 | 9.41 | 6.663619 |
| 8 | 201 | 2.59 | 9.42 | 5.244759 |
| 9 | 242 | 2.59 | 9.42 | 6.314585 |
| 10 | 206 | 2.59 | 9.42 | 5.369525 |
| Average | 231.4 | 2.61 | 9.422 | 5.987306 |
| Std. dev. | 20.30435 | 0.0290593 | 0.006325 | 0.478592 |

TABLE IV
Compression Force
6.35 kN

| | Hardness | Thickness | Diameter | Tensile |
|---|---|---|---|---|
| Tablet # | (N) | (mm) | (mm) | Strength (MPa) |
| 1 | 133 | 2.83 | 9.45 | 3.166019 |
| 2 | 141 | 2.86 | 9.44 | 3.324767 |
| 3 | 158 | 2.87 | 9.43 | 3.716581 |
| 4 | 114 | 2.81 | 9.44 | 2.735941 |
| 5 | 121 | 2.78 | 9.43 | 2.938387 |
| 6 | 119 | 2.83 | 9.43 | 2.838762 |
| 7 | 124 | 2.82 | 9.42 | 2.971678 |
| 8 | 135 | 2.82 | 9.43 | 3.231864 |
| 9 | 141 | 2.84 | 9.43 | 3.351731 |
| 10 | 134 | 2.81 | 9.43 | 3.21934 |
| Average | 132 | 2.827 | 9.433 | 3.149507 |
| Std. dev. | 13.0384 | 0.0258414 | 0.008233 | 0.288465 |

TABLE V
Compression Force
15.55kN

| | Hardness | Thickness | Diameter | Tensile |
|---|---|---|---|---|
| Tablet # | (N) | (mm) | (mm) | Strength (MPa) |
| 1 | 245 | 2.52 | 9.43 | 6.563477 |
| 2 | 278 | 2.53 | 9.41 | 7.433867 |
| 3 | 259 | 2.51 | 9.42 | 6.973572 |
| 4 | 261 | 2.53 | 9.42 | 6.971869 |
| 5 | 253 | 2.46 | 9.41 | 6.957864 |
| 6 | 241 | 2.53 | 9.41 | 6.444467 |
| 7 | 255 | 2.52 | 9.41 | 6.845894 |
| 8 | 238 | 2.45 | 9.42 | 6.565081 |
| 9 | 239 | 2.55 | 9.41 | 6.340861 |
| 10 | 266 | 2.48 | 9.42 | 7.248684 |
| Average | 253.5 | 2.508 | 9.416 | 6.834563 |
| Std. dev. | 13.01495 | 0.0332666 | 0.006992 | 0.353741 |

TABLE VI
Compression Force
8.79 kN

| | Hardness | Thickness | Diameter | Tensile |
|---|---|---|---|---|
| Tablet # | (N) | (mm) | (mm) | Strength (MPa) |
| 1 | 207 | 2.66 | 9.47 | 5.236941 |
| 2 | 193 | 2.74 | 9.43 | 4.935394 |
| 3 | 188 | 2.75 | 9.43 | 4.789392 |
| 4 | 181 | 2.62 | 9.43 | 4.663862 |
| 5 | 169 | 2.63 | 9.43 | 4.338098 |
| 6 | 181 | 2.61 | 9.42 | 4.686701 |
| 7 | 195 | 2.68 | 9.42 | 4.917326 |
| 8 | 173 | 2.6 | 9.44 | 4.487256 |
| 9 | 200 | 2.66 | 9.43 | 5.075944 |
| 10 | 164 | 2.59 | 9.43 | 4.274768 |
| Average | 185.1 | 2.634 | 9.432 | 4.740568 |
| Std. dev. | 13.96384 | 0.0291357 | 0.011353 | 0.313112 |

### Example 6

### Piroxicam:ProSolv®

In this example, slurry coprocessing of Piroxicam with hydro-cellulose was carried out to see the effects hydrocellulose has on the Piroxican functional characteristics.

| Materials: | Piroxicam | |
|---|---|---|
| | Temalfa T 1 Slurry (rapidly hydrating cellulose) | |
| | Collodial Silicon Dioxide | |
| Equipment: | Niro Production Minor Spray Dryer | |
| | Balance #0173 | |
| | Horiba Particle Size Analyzer | |
| Methods: | As described above in the methods above | |
| Data: | Batch Size | 2 kg |
| | Required Slurry | 7.68 kgs (78%) |
| | Required CSD | 0.01 kg (2%) |
| | Required Piroxicam | 0.05 kg (25%) |
| | Solids Content | 19.25% |
| | Inlet Temp | 200° C. |
| | Outlet Temp | 100° C. |
| | Feed Rate | 5 |
| | Atomizer Freq | 45 Hz |
| | Particle Size | 48 μm |

### CONCLUSION

During the initial mixing it became apparent that more water would be required to slurry the Piroxicam with the Temalfa T1 Slurry. An additional 2 kg of water was added which lowered the cellulose solids to 15.08% (19%) and the total solids content to 19.25% (23.95%), these changes produced a workable slurry. The slurry was then spray dried without any issues, particle size was achieved.

### Example 7

### Piroxicam:ProSolv®

In this example slurry co-processed Piroxicam with sodium lauryl sulfate agglomerated particles were prepared.

| Materials: | Piroxicam | |
|---|---|---|
| | ProSolv 50 (98% microcrystalline cellulose:2% | |
| | colloidal silicon dioxide) | |
| | Sodium Lauryl Sulfate | |
| Equipment: | Niro Production Minor Spray Dryer | |
| | Balance #0173 | |
| | Horiba Particle Size Analyzer | |
| Methods: | As described in the methods above. | |
| Data: | Batch Size | 2.5 kg |
| | Piroxicam % | 25% |
| | Required Piroxicam | 0.625 |
| | ProSolv % | 73% |
| | Required ProSolv | 1.825 |
| | SLS % | 0.5% |
| | Required SLS | .0125 |
| | Required H2O | 7.71 kg |
| | Inlet Temp | 240° C. |
| | Outlet Temp | 99° C. |
| | Feed Rate | 15 |
| | Atomizer Freq | 25 Hz |
| | Gas Pressure | 6.9″ H2O |
| | Solids Content | 7.50% |
| | Particle Size | 38 μm |

### Examples 8-11

### Piroxicam Tablets

### Example 8

In this example, Piroxicam tablets were prepared (Formula 5) having the following ingredients:

| | | |
|---|---|---|
| Materials: | Co-processed Piroxicam | 97.75% |
| | (25% Piroxicam:75% ProSolv ®) of Example 4 | |
| | Vivasol (Croscarmellose sodium) | 2% |
| | Pruv (Sodium Stearyl Fumarate) | 0.25% |

### Example 9

In this example, Piroxicam tablets were prepared (Formula 1) having the following ingredients:

| | | |
|---|---|---|
| Materials: | Co-processed Piroxicam | 97.50% |
| | (25% Piroxicam:75% ProSolv ®) of Example 4 | |
| | Vivasol (Croscarmellose sodium) | 2% |
| | Sodium Lauryl Sulfate (SLS) | 0.25% |
| | Pruv (Sodium Stearyl Fumarate) | 0.25% |

### Example 10

In this example, Piroxicam tablets were prepared (Formula 2) having the following ingredients:

| | | |
|---|---|---|
| Materials: | Co-processed Piroxicam | 24.67% |
| | (25% Piroxicam:75% ProSolv ®) of Example 4 | |
| | HD ProSolv ® | 73.08% |
| | Vivasol (Croscarmellose sodium) | 2% |
| | Pruv (Sodium Stearyl Fumarate) | 0.25% |

### Example 11

In this example, Piroxicam tablets were prepared (Formula 4) having the following ingredients:

| | | |
|---|---|---|
| Materials: | Co-processed Piroxicam | 24.67% |
| | (25% Piroxicam:75% ProSolv ®) of Example 4 | |
| | HD ProSolv ® | 72.83% |
| | Vivasol (Croscarmellose sodium) | 2% |
| | Sodium Lauryl Sulfate (SLS) | 0.25% |
| | Pruv (Sodium Stearyl Fumarate) | 0.25% |

### Example 12

In this example, Piroxicam tablets were prepared (Formula 5) having the following ingredients:

| | | |
|---|---|---|
| Materials: | Piroxicam (non-coprocessed) | 4.66% |
| | HD ProSolv ® | 93.09% |
| | Vivasol (Croscarmellose sodium) | 2% |
| | Pruv (Sodium Stearyl Fumarate) | 0.25% |

### Example 13

In this example, Piroxicam tablets were prepared (Formula 6) having the following ingredients:

| | | |
|---|---|---|
| Materials: | Piroxicam (non-coprocessed) | 4.66% |
| | HD ProSolv ® | 92.84% |
| | Vivasol (Croscarmellose sodium) | 2% |
| | Sodium Lauryl Sulfate (SLS) | 2% |
| | Pruv (Sodium Stearyl Fumarate) | 0.25% |

### Examples 14-24

### Glucosamine Co-Processing

### Example 14

### Glucosamine:ProSolv®

In this example Glucosamine CP was co-processed using the same configurations as the Regenasure co-processing. The overall ProSolv level was lowered from 21% to 18%.

| Materials: | Glucosamine CP | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Haribe Particle Size Analyzer | |
| Methods: | As described above in the methods above | |
| Data: | Batch Size | 8 kg |
| | Glucosamine % | 82% |
| | Required Glu | 6.56 kg |
| | ProSolv % | 18% |
| | Required ProSolv | 1.44 kg |
| | Solids Content | 40% |
| | Inlet Temp | 240° C. |
| | Outlet Temp | 99° C. |
| | Feed Rate | 5 |
| | Atomizer Freq | 50 Hz |
| | Gas Pressure | 6.9″ H2O |

### CONCLUSION

Material was bagged and collected. An in process particle size analysis was performed and the median particle size came out around 52 μm, the atomizer wheel was slowed down in an attempt to increase particle size. Particle size was run again at the end of the run and despite slowing the wheel down the median particle size stayed around 50 μm.

### Example 15

### Co-Processed Glucosamine CP/Chondroitin/MSM

In this example, co-processed Glucosamine was blended with chondroitin and MSM.

| | |
|---|---|
| Materials: | Coprocessed Glucosamine CP (18%) |
| | Chondroitin Shengguan 6409-14 |
| | MSM 04032.10 |
| | Em 608oy P510000340 |
| | Talc QI 0912 |
| | Magnesium Sterate ND0154 |
| Equipment: | Patterson Kelly V Blender |
| | 5 qt V Blender Shell |
| | Balance #0275 |
| | 20 Mesh Sieve |
| Methods: | As described above in the methods above |
| Blend Data: | 750 g |

| | |
|---|---|
| Glucosamine | 43.07% = 323 |
| Chondroitin | 28.74% = 223 |
| MSM | 21.19% = 158.9 |
| Emcosoy | 4.00% = 30 |
| Talc | 1.50% = 11.3 |
| Magneisum Sterate | .50% = 3.8 |

Total Blend Time = 30 minutes

### Example 16

### Co-Processed Glucosamine (18%)

A newly produced co-processed Glucosamine (18%) was blended. This co-processed Glucosamine was tested to see if lowering the ProSolv level during the co-processing impacts the functionality.

| Material: | 1079-126 | |
|---|---|---|
| Equipment: | Korsch PH 106 Rotary Tablet Press | |
| | Erweka TBH-30 | |
| | Balance # 0274 | |
| | Hygrometer # | |
| Methods: | Press Speed | 50 rpm |
| | Punch Size | Hob #6212 |
| | Target Weight | 1415 (t5) |
| | Compaction Forces | 30, 35 KW |

### CONCLUSION

The blend was run and the hardness values were 25 kp at 30 kn and 27 kp at 35 kn. These values represent a significant increase over the previous formulation produced with the 18% co-processed Regenasure Glucosamine (16 kp). At the moment there is no reason to explain the bump in hardness. A few dry powder addition blends with ProSolv will be made and tableted to see if that any insight.

### Example 17

### Glucosamine CP/Custom Grade ProSolv® (5%)

In this example, Glucosamime CP was co-processed with a ProSolv® custom grade (5%) to determine if the custom grade of ProSolv® provides any benefit in terms of compaction.

| Materials: | Glucosamine CP 200463127 | |
|---|---|---|
| | ProSolv Custom Grade (5%) | |
| Equipment: | Niro Production Miner Spray Dryer | |
| | Balance #0173 | |
| | Haribs Particle Size Analayzer | |
| Methods: | As described above in the methods above | |
| Data: | Batch Size | 2 kg |
| | Gluocsamine % | 82% |
| | Required Gluocsamine | 1.64 kg |
| | ProSolv % | 18% |
| | Required ProSolv | .36% |
| | Solids Content | 40% |
| | Inlet Temp | 240° C. |
| | Outlet Temp | 99° C. |
| | Feed Rate | 7 |
| | Atomizer Freq | 50 Hz |
| | Gas Pressure | 6.8″ H2O |
| | Particle Size | 69 μm |

### CONCLUSION

Run went smoothly, no problems occurred. Material labeled and bagged.

### Example 18

### Glucosamine CP/Custom Grade ProSolv® (5%)

In this example, a blend of coprocessed Glucosamine made with custom grade ProSolv (5%) was prepared.

| | |
|---|---|
| Materials: | Co Processed Gluocsamine |
| | Chondroitin Shengguan 0409-14 |
| | Emcosoy PS 10000340 |
| | Talc QI 0912 |
| | Magnesium Sterate Nd0154 |
| Equipment: | Patterson Kelly V Blender |
| | 5 qt V Blender Shell |
| | Balance #0275 |
| | 20 Mesh Sieve |
| Methods: | As described above in the methods above |
| Blend Data: | 1000 g |

| | |
|---|---|
| Glucosamine | 54.58% = 545.3 |
| Chondroitin | 89.47% = 394.7 |
| Emcosoy | 4.00% = 40 |
| Talc | 1.50% = 15 |
| Magnesium Sterate | .50% = 5 |

### Example 19

### Compaction Comparison

In this example, Glucosamine blends were compacted in order to see if the added CSD (ProSolv Custom Grade) enhanced the compaction of the coprocessed Glucosamine.

| | |
|---|---|
| Material: | 1079-187 (5% CSi) |
| Equipment: | Korsch Plt 106 Rotary Tablet Press |
| | Erweka TBH-30 |
| | Balance #0274 |
| | Hygrometer #0526 |
| Methods: | As described above in the methods above |

| | |
|---|---|
| Press Speed | 50 rpm |
| Punch Size | Hob#6212 |
| Target Weight | 1192 (+/−5 mg) |
| Compaction Forces | 30, 35 kN |

### CONCLUSION

Another blend of coprocessed Glucosamine without the extra CSD was run to compare the Custom Grade co processed Glucosamines compactibility. Despite the extra CSD the compaction value was 3 kp lower at 30 Kn and 6 kp at 35 kN.

### Example 20

In this example, an earlier attempt was made at coprocessing Glucosamine in order to produce a 65 cm material for customer evaluation. The material produced had a median particle size around 83 μm. This material was tested and the increase in particle size had no negative effects on compaction. Another attempt was made to produce material closer to 65 μm for customer evaluation.

| Materials: | Glucosamine CP 20040327 (NatureGen Inc.) | |
|---|---|---|
| | ProSolv 50 (98% microcrystalline cellulose: 2% | |
| | colloidal silicon dioxide) | |
| Equipment: | Miro Production Minor Spray Dryer | |
| | Balance #0173 | |
| | Horiba Particle Size Analyzer | |
| Methods: | As described above in the methods above | |
| Data: | Batch Size | 10 kg |
| | Glucosamine % | 82% |
| | Req Gluocsamine | 4.1 kg |
| | ProSolv % | 18% |
| | Req ProSolv | .9 kg |
| | Solids Content | 40% |
| | Inlet Temp | 240° C. |
| | Outlet Temp | 99° C. |
| | Feed Rate | 13 |
| | Atomizer Freq | 50 Hz/40 Hz |
| | Gas Pressure | 6.9″ H2O |
| | Particle Size | 54 μm/65 μm |

### CONCLUSION

The run was split into two 5 kg runs in case any changes needed to be made. The first 5 kg run was run at 50 Hz and produced material around 54 μm. The atomizer was slowed down to 40 Hz for the second half of the run and it increased the particle size to 65 μm. The material was bagged and samples were pulled for internal testing on the material.

### Example 21

A new batch of coprocessed Glucosamine was made for evaluation.

| Material: | Glucosamine/Chondroitin Blend | |
|---|---|---|
| Equipment: | Korsch PH106 Rotary Tablet Press | |
| | Balance #0274 | |
| | Erweka TBH-30 | |
| | Hygrometer #0526 | |
| Methods: | As described above in the methods above | |
| Data: | Press Speed | 50 rpm |
| | Punch Size | Hob#6212 |
| | Target Weight | 1192 (+/−5) |
| | Compaction Force | 35 kN |

### CONCLUSION

Testing confirms that the material was within spec. range (20 kp).

### Example 22

In this example, a CP 23 Glucosamine was co-processed.

| Materials: | Regenasure Glucosamine RUE4088A (HCL) | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | 10 kg |
| | Glucosamine % | 77% |
| | Required Glucosamine | 7.7 kg |
| | ProSolv % | 23% |
| | Required ProSolv | 2.3 kg |
| | Required H2O | 11.55 |
| | Solids Content | 40% |
| | Inlet Temp | 240° C. |
| | Outlet Temp | 99° C. |
| | Feed Rate | 17 |
| | Atomizer Freq | 50 Hz |
| | Gas Pressure | 6.9″ H2O |
| | Particle Size | 63 μm |

### CONCLUSION

Spray Drying run was completed, and a sample retained.

### Example 23

In this example, 25-30 kgs of coprocessed Glucosamine was produced. In order to produce 25-30 kgs, the batch size had to be 10 kg at a time. This was done to prevent large buildup of material on the walls of the spray dryer. The same configurations were used to produce two separate 10 kg batches.

| Materials: | Regensure HCl GLucosamine RUE 4088A | |
|---|---|---|
| | ProSolv 50 (98% microcrystalline cellulose:2% | |
| | colloidal silicon dioxide) | |
| Equipment: | Niro Production Minor Spray Dryer | |
| | Balance #0173 | |
| | Horiba Particle Size Analyzer | |
| Methods: | As described above in the methods above | |
| Data: | Batch Size | 10 kg |
| | Glucosamine % | 77% |
| | Required Glucosamine | 7.7 kg |
| | ProSolv % | 23% |
| | Required ProSolv | 2.3 kg |
| | Required H2O | 11.55 |
| | Solids Content | 40% |
| | Inlet Temp | 240° C. |
| | Outlet Temp | 99° C. |
| | Feed Rate | 16 |
| | Atomizer Freq | 50 Hz |
| | Gas Pressure | 6.9″ H2O |
| | Particle size | 65 μm |

### Example 24

In this example, two new coprocessed Glucosamine batches were produced for testing. A blend of the standard 500/400 Glucosamine/Chondroitin formulation for each co-processed Glucosamine was prepared.

| Equipment: | Korsch PH 106 Rotary Tablet Press | |
|---|---|---|
| | Erweka TBH-30 | |
| | Balance #0274 | |
| | Hygrometer #0526 | |
| Methods: | As described above in the methods above | |
| Data: | Press Speed | 50 rpm |
| | Punch Size | Hob#6212 (.75 × .3125) |
| | Target Weight | 1192 (+/−5) |
| | Compaction Forces | 30, 35 |

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A method of preparing a pharmaceutical formulation, comprising:
a) preparing a slurry of pre-manufactured agglomerated particles consisting of microcrystalline cellulose and one or more compressibility augmenting agents, and an active agent; and
b) drying the slurry to form active agent agglomerated particles.

2. A method of preparing a pharmaceutical formulation, comprising:
a) preparing an aqueous slurry of microcrystalline cellulose, a compressibility augmenting agent and other, optional, pharmaceutically acceptable excipients;
b) drying the mixture of ingredients prepared in step a) in a manner which inhibits quasi-hornification of the microcrystalline cellulose to obtain agglomerate particles;
c) preparing a slurry containing the agglomerated particles obtained in step b) together with a suitable amount of an active agent, and other, optional, pharmaceutically acceptable excipients; and;
d) drying the slurry to form active agent agglomerated particles.

3. The method of claim 1, wherein the compressibility augmenting agent
(i) physically restricts the proximity of the interface between adjacent cellulose surfaces; or
(ii) inhibits interactions between adjacent cellulose surfaces; or
(iii) both (i) and (ii).

4. The method of claim 3, wherein the compressibility augmenting agent is selected from the group consisting of a highly polar molecule in an amount effective to augment the compressibility of the microcrystalline cellulose, a surfactant and any combination or mixture thereof.

5. The method of claim 4, wherein the compressibility augmenting agent is a silicon dioxide having an average primary particle size from about 1 nm to about 100 μm.

6. The method of claim 4, wherein the compressibility augmenting agent is colloidal silicon dioxide.

7. The method of claims 5, wherein the silicon dioxide is included in amount from about 0.1% to about 20% by weight, based on the weight of microcrystalline cellulose.

8. The method of claim 5, wherein said silicon dioxide is included in an amount of from about 1.25% to about 5%, based on the weight of said microcrystalline cellulose.

9. The method of claim 5, wherein the silicon dioxide portion of the agglomerate is derived from a silicon dioxide having a surface area from about 10 m²/g to about 500 m²/g.

10. The method of claim 5, wherein the compressibility augmenting agent comprises effective amounts of a silicon dioxide having an average primary particle size from about 1 nm to about 100 μm and a surfactant having an HLB value from about 15 to about 50.

11. The method of claim 1, wherein the active agent agglomerated particles have an average particle size of from about 10 μm to about 300 μm; preferably from 30 μm to about 125 μm; and more preferably about μm 65.

12. The method of claim 5, wherein said silicon dioxide is derived from a silicon dioxide having a surface area from about 175 m²/g to about 350 m²/g.

13. The method of claim 1, wherein the active agent is selected from the group consisting of Piroxicam, pharmaceutically acceptable salts, derivatives and mixtures thereof.

14. The method of claim 1, wherein the active agent is selected from the group consisting of Glucosamine, pharmaceutically acceptable salts, esters, derivatives and mixtures thereof.

15. The method of claim 1, wherein the active agent is selected from the group consisting of chondroitin, pharmaceutically acceptable salts, esters, derivatives and mixtures thereof.

16. The method of claim 1, wherein the formulation contains a combination of active agents.

17. The method of claim 16, wherein the formulation contains a combination of glucosamine and chondroitin and any pharmaceutically acceptable salts, esters, derivatives or mixtures thereof.

18. The method of claim 1, wherein a surfactant is added to the slurry.

19. The method of claim 1, wherein a surfactant is dried together with the slurry by introducing the surfactant into the dryer separately from the slurry.

20. The method of claim 18, wherein the surfactant is sodium lauryl sulfate.

21. The method of claim 1, wherein one or more additional pharmaceutically acceptable excipients are added to the slurry prior to drying.

22. The method of claim 1, wherein one or more additional pharmaceutically acceptable excipients are dried together with the slurry by introducing the excipient into the dryer separately from the slurry.

23. The method of claim 21, wherein the one or more additional pharmaceutically acceptable excipients is selected from the group consisting of binders, diluents, disintegrators, lubricants, preserving agents, fillers, surfactants and wetting agents, emulsifying agents, suspending agents, sweetening agents, flavoring agents, perfuming agents, dispensing agents and any combinations or mixtures thereof.

24. The method of claim 1, further comprising the step of incorporating the active agent agglomerated particles into a solid dosage form.

25. The method of claim 24, wherein the active agent agglomerated particles are compressed into a tablet.

26. The method of claim 24, wherein the active agent agglomerated particles are incorporated into a capsule.

27. The method of claim 25, wherein the dosage form is selected from the group consisting of an immediate release dosage form, a delayed release dosage form, a sustained release dosage form, a bi-modal release dosage form, a pulsatile release dosage form or any combinations thereof.

28. The method of claim 1, wherein the active agent is a wetted active agent.

29. The method of claim 1, wherein the active agent to pre-manufactured agglomerated particles is from about 90:10 to about 10:90; preferably 60:40 to 40:60; more preferably 75:25 to 25:75.

30. The method of claim 1, wherein the slurry has a solids content of from about 1% to about 40%; preferably 5% to 25%.

31. The method of claim 1, wherein the cellulose content of the slurry is about 14% to about 24%.

32. The method of claim 18, wherein the amount of surfactant is from about 0.01% to about 5%.

33. The method of claim 1, wherein the slurry is dried in a spray-dryer.

34. A method of preparing a pharmaceutical formulation, comprising:
a) preparing a slurry of pre-manufactured agglomerated particles consisting of microcrystalline cellulose and one or more compressibility augmenting agents; and
b) combining dry active agent particles and the slurry in a dryer to form active agent agglomerated particles.

35. A method of preparing a pharmaceutical formulation, comprising:
a) preparing a slurry of microcrystalline cellulose, an active agent and a compressibility augmenting agent, wherein the
compressibility augmenting agent (i) physically restricts the proximity of the interface between adjacent cellulose surfaces; or (ii) inhibits interactions; and
b) drying the slurry to form active agent agglomerated particles.

36. The methods of claim 34, further comprising the step of incorporating the active agent agglomerated particles into a solid dosage form.
